(19) United States
(12) Patent Application Publication
(10) Pub. No.: US 2026/0272079 A1
(43) Pub. Date: Sep. 17, 2026

(54) MEDICAL GOWN OR BODY SUIT FOR CONNECTED CARE

(71) Applicant: Hill-Rom Services, Inc., Batesville, IN (US)

(72) Inventors: Jnanesha RAMEGOWDA, Batesville, IN (US); Douglas E. BORGMAN, Brookville, IN (US); Darrell L. BORGMAN, Batesville, IN (US); Kathryn Ruth SMITH, Batesville, IN (US); Timothy Michael VOLK, Harrison, OH (US); Frank E. SAUSER, Cincinnati, OH (US); Jeffrey HOOG, Sunman, IN (US); Taylor VOHLAND, Batesville, IN (US)

(21) Appl. No.: 19/566,123

(22) Filed: Mar. 13, 2026

Related U.S. Application Data
(60) Provisional application No. 63/772,353, filed on Mar. 14, 2025.

(51) Int. Cl. A41D 13/12; A41D 13/002; A41D 13/005; A61F 7/00

(52) U.S. Cl. CPC A41D 13/1281; A41D 13/0025; A41D 13/0051; A41D 13/0053; A61F 7/007; A61F 2007/0096

(57) ABSTRACT

This disclosure relates to devices and methods to improve patient care by increasing patient monitoring and data acquisitions. More specifically, this disclosure relates to wearable devices that can be used to monitor patients to obtain information including but not limited to patient movement, patient therapy, patient physiological measurements, and patient behavior.

### PRIORITY CLAIM

This application claims priority under 35 U.S.C. § 119(e) to U.S. Provisional Application No. 63/772,353, filed Mar. 14, 2025, which is expressly incorporated by reference herein.

### BACKGROUND

The present disclosure relates to the use of systems and methods to improve patient care by optimizing use and efficiency of medical gowns or body suits. Physicians can provide improved diagnoses and better patient care if they are able to monitor patients at all times. Continuous monitoring increases the odds of capturing sporadic conditions, during which a patient can also engage in activities outside the patient support apparatus. With extended ambulatory monitoring in-clinic diagnostic efficacy and patient outcome can be improved.

Typically monitoring devices and/or sensors are attached to the patient support apparatus or are located in patient rooms. When a monitoring device is moved around a hospital a lot of maneuvering may be required, and the patient may be out of the range of such monitoring devices and/or sensors at different times. Further, if the patient exits the patient room or is separated from the patient support apparatus, patient monitoring is interrupted, thereby affecting therapeutic functions associated with patient monitoring.

The high cost of the patient-wearable components used to provide extended monitoring can also negatively influence the availability and use of monitors. Disposable components, such as adhesive electrodes, ideally should be inexpensive, while more complex components, particularly the electronic hardware that detects and records signals and related physiological data, may be unavoidably expensive. Costs can be balanced by designing the electric hardware to be re-usable, but when the total cost of a full ECG monitoring ensemble remains high, despite the utilization of re-usable parts, the number of monitors available for use by healthcare providers can be inhibited. Cost, then, becomes a barrier to entry, which, in turn, can hinder or prevent healthcare providers from obtaining the means with which to efficaciously identify the physiology underlying sporadic cardiac arrhythmic conditions and can ultimately contribute to a failure to make proper and timely medical diagnoses.

This disclosure relates to accessories for patient support apparatus. Specifically, this disclosure relates to wearable devices that can be used to monitor patient data including but not limited to patient movement, patient physiological measurements, and patient behavior.

### SUMMARY

The present disclosure includes one or more of the features recited in the appended claims and/or the following features which, alone or in any combination, may comprise patentable subject matter.

According to a first aspect, the present disclosure is directed to a garment comprising a disposable portion comprising a monitoring system having a sensor system is configured to provide a signal, a reusable portion, the reusable portion comprising a control system having a control unit with a processor configured to receive and analyze the signal from the disposable portion and a power source configured to power the control unit, and wherein, the monitoring system is configured to provide data about patient condition, a patient therapy, or an environmental condition associated with the patient.

In some embodiments of the first aspect, the garment of claim 1, the sensor system may be charged by the power source located in the reusable portion of the garment.

In some embodiments of the first aspect, the garment of claim 1, the sensor system may be configured to provide microclimate environment data to the control unit, and wherein the control unit is configured to direct a blower connected to the control unit to blow air through a plurality of air channels in the disposable portion of the garment.

In some embodiments of the first aspect, the disposable portion may comprise a 3D spacer material configured to create the plurality of air channels.

In some embodiments of the first aspect, the blower may blow hot air or cold air through the plurality of air channels.

In some embodiments of the first aspect, the garment may comprise a plurality of zones, and wherein the blower may be configured to blow air through one or more zones.

In some embodiments of the first aspect, the blower may be configured to blow air through the plurality of air channels in the garment in response to a user input provided at a user interface of the control unit.

In some embodiments of the first aspect, the sensor system may be a wound sensor system configured to provide wound healing data to the control unit, and wherein the control unit is configured to direct a heating element located in the wound sensor system.

In some embodiments of the first aspect, the wound sensor system may be configured to measure temperature, blood flow, oxygen level, moisture content, pH, or depth of a wound.

In some embodiments of the first aspect, the wound sensor system may include the heating element positioned on a wound facing side of a wound dressing.

In some embodiments of the first aspect, the disposable portion may comprise one or more patches of foam configured to minimize abrasion in zones that cover parts of patient body prone to wounds.

In some embodiments of the first aspect, the disposable portion may comprise a first layer separated from a second layer by a layer of hydrogel.

In some embodiments of the first aspect, the sensor system may be configured to measure a height of the layer of hydrogel.

In some embodiments of the first aspect, the control unit may be configured to determine a change in the height of the layer of hydrogel compared to a baseline measurement and indicate an incontinence event if the height of the layer of hydrogel exceeds the baseline measurement.

In some embodiments of the first aspect, the sensor system may comprise a RFID tag configured to indicate an incontinence event.

In some embodiments of the first aspect, the garment may comprise a movement monitoring system that includes the sensor system in a movement detection system configured to provide movement associated data with at least one part of patient body.

In some embodiments of the first aspect, the movement detection system may comprise a voltage sensor, a capacitive sensor, or an accelerometer.

In some embodiments of the first aspect, the movement detection system may comprise a fabric that responds to heat or voltage.

In some embodiments of the first aspect, the movement detection system may comprise an accelerometer sewn to the garment.

In some embodiments of the first aspect, the garment may comprise a tracing system wherein the sensor system comprises a tracking device configured to provide patient location data.

In some embodiments of the first aspect, tracking device may be located on a wrist band, an adhesive patch, an ear bud, an ankle bracelet, or a head covering of the garment.

In some embodiments of the first aspect, the garment may comprise a vital monitoring system, wherein the sensor system is configured to provide patient health data.

In some embodiments of the first aspect, the sensor system may comprise a temperature sensor, a moisture sensor, a pH sensor, a heart rate monitor, or an oxygen monitor.

In some embodiments of the first aspect, the garment may comprise a light therapy system that includes a one or more lights or LEDs configured to provide light to different parts of a patient body.

In some embodiments of the first aspect, the light from the one or more lights or LEDs may be of the same wavelength.

In some embodiments of the first aspect, the light from the one or more lights or LEDs may be of different wavelengths.

According to a second aspect, the present disclosure is directed to a garment comprising a disposable portion comprising a sensor system configured to provide a signal, a reusable portion, the reusable portion comprising a control system having a control unit with a processor configured to receive and analyze the signal from the disposable portion and a power source configured to power the control unit, and wherein, the sensor system is configured to provide data to a predictive AI system.

In some embodiments of the second aspect, the data may be about patient condition, a patient therapy, or an environmental condition associated with the patient.

In some embodiments of the second aspect, the sensor system may be charged by the power source located in the reusable portion of the garment.

In some embodiments of the second aspect, the sensor system may be configured to provide microclimate environment data to the control unit, and wherein the control unit may be configured to direct a blower connected to the control unit to blow air through a plurality of air channels in the disposable portion of the garment.

In some embodiments of the second aspect, the disposable portion may comprise a 3D spacer material configured to create the plurality of air channels.

In some embodiments of the second aspect, the blower may be configured to blow hot air or cold air through the plurality of air channels.

In some embodiments of the second aspect, the garment may comprise a plurality of zones, and the blower may be configured to blow air through one or more zones.

In some embodiments of the second aspect, the blower may be configured to blow air through the plurality of air channels in the garment in response to a user input provided at a user interface of the control unit.

In some embodiments of the second aspect, the sensor system may be a wound sensor system configured to provide wound healing data to the control unit, and wherein the control unit may be configured to direct a heating element located in the wound sensor system.

In some embodiments of the second aspect, the wound sensor system may be configured to measure temperature, blood flow, oxygen level, moisture content, pH, or depth of a wound.

In some embodiments of the second aspect, the wound sensor system may comprise the heating element positioned on a wound facing side of a wound dressing.

In some embodiments of the second aspect, the disposable portion may comprise one or more patches of foam configured to minimize abrasion in zones that cover parts of patient body prone to wounds.

In some embodiments of the second aspect, the disposable portion may comprise a first layer separated from a second layer by a layer of hydrogel.

In some embodiments of the second aspect, the sensor system may be configured to measure a height of the layer of hydrogel.

In some embodiments of the second aspect, the control unit may be configured to determine a change in the height of the layer of hydrogel compared to a baseline measurement and indicate an incontinence event if the height of the layer of hydrogel exceeds the baseline measurement.

In some embodiments of the second aspect, the sensor system may comprise a RFID tag configured to indicate an incontinence event.

In some embodiments of the second aspect, the garment may comprise a movement monitoring system that includes the sensor system in a movement detection system configured to provide movement associated data with at least one part of patient body.

In some embodiments of the second aspect, the movement detection system may comprise a voltage sensor, a capacitive sensor, or an accelerometer.

In some embodiments of the second aspect, the movement detection system may comprise a fabric that responds to heat or voltage.

In some embodiments of the second aspect, the movement detection system may comprise an accelerometer sewn to the garment.

In some embodiments of the second aspect, the garment may comprise a tracing system wherein the sensor system comprises a tracking device configured to provide patient location data.

In some embodiments of the second aspect, the tracking device may be located on a wrist band, an adhesive patch, an ear bud, an ankle bracelet, or a head covering of the garment.

In some embodiments of the second aspect, the garment may comprise a vital monitoring system, wherein the sensor system may be configured to provide patient health data.

In some embodiments of the second aspect, the sensor system may comprise a temperature sensor, a moisture sensor, a pH sensor, a heart rate monitor, or an oxygen monitor.

In some embodiments of the second aspect, the garment may comprise a light therapy system that includes a one or more lights or LEDs configured to provide light to different parts of a patient body.

In some embodiments of the second aspect, the light from the one or more lights or LEDs may be of the same wavelength.

In some embodiments of the second aspect, the light from the one or more lights or LEDs may be of different wavelengths.

According to a third aspect, the present disclosure is directed to a method of acquiring data about a patient condition, a patient therapy, or an environmental condition associated with a patient comprising, monitoring the patient wearing a garment, the garment including a disposable portion and comprising a monitoring system having a sensor system, and a reusable portion comprising a control system including a control unit and a power source, acquiring a signal from the sensor system located on the garment, processing the signal in the control system with a processor configured to receive and analyze the signal, and outputting processed data to a caregiver

In some embodiments of the third aspect, the sensor system may be charged by the power source located in the reusable portion of the garment.

In some embodiments of the third aspect, the sensor system may be configured to provide microclimate environment data to the control unit, and wherein the control unit may be configured to direct a blower connected to the control unit to blow air through a plurality of air channels in the disposable portion of the garment.

In some embodiments of the third aspect, the disposable portion may comprise a 3D spacer material configured to create the plurality of air channels.

In some embodiments of the third aspect, the blower may be configured to blow hot air or cold air through the plurality of air channels.

In some embodiments of the third aspect, the garment may comprise a plurality of zones and the blower may be configured to blow air through one or more zones.

In some embodiments of the third aspect, the blower may be configured to blow air through the plurality of air channels in the garment in response to a user input provided at a user interface of the control unit.

In some embodiments of the third aspect, the sensor system may be a wound sensor system configured to provide wound healing data to the control unit, and wherein the control unit is configured to direct a heating element located in the wound sensor system.

In some embodiments of the third aspect, the wound sensor system may be configured to measure temperature, blood flow, oxygen level, moisture content, pH, or depth of a wound.

In some embodiments of the third aspect, the wound sensor system may comprise the heating element positioned on a wound facing side of a wound dressing.

In some embodiments of the third aspect, the disposable portion may comprise one or more patches of foam configured to minimize abrasion in zones that cover parts of patient body prone to wounds.

In some embodiments of the third aspect, the disposable portion may comprise a first layer separated from a second layer by a layer of hydrogel.

In some embodiments of the third aspect, the sensor system may be configured to measure a height of the layer of hydrogel.

In some embodiments of the third aspect, the control unit may be configured to determine a change in the height of the layer of hydrogel compared to a baseline measurement and indicate an incontinence event if the height of the layer of hydrogel exceeds the baseline measurement.

In some embodiments of the third aspect, the sensor system may comprise a RFID tag configured to indicate an incontinence event.

In some embodiments of the third aspect, the garment may comprise of a movement monitoring system that includes the sensor system in a movement detection system configured to provide movement associated data with at least one part of patient body.

In some embodiments of the third aspect, the movement detection system may comprise a voltage sensor, a capacitive sensor, or an accelerometer.

In some embodiments of the third aspect, the movement detection system may comprise a fabric that responds to heat or voltage.

In some embodiments of the third aspect, the movement detection system may comprise an accelerometer sewn to the garment.

In some embodiments of the third aspect, the garment may comprise a tracing system wherein the sensor system comprises a tracking device configured to provide patient location data.

In some embodiments of the third aspect, the tracking device may be located on a wrist band, an adhesive patch, an ear bud, an ankle bracelet, or a head covering of the garment.

In some embodiments of the third aspect, the garment may comprise a vital monitoring system, wherein the sensor system is configured to provide patient health data.

In some embodiments of the third aspect, the sensor system may comprise a temperature sensor, a moisture sensor, a pH sensor, a heart rate monitor, or an oxygen monitor.

In some embodiments of the third aspect, the garment may comprise a light therapy system that includes a one or more lights or LEDs configured to provide light to different parts of a patient body.

In some embodiments of the third aspect, the light from the one or more lights or LEDs may be of the same wavelength.

In some embodiments of the third aspect, the light from the one or more lights or LEDs may be of different wavelengths.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is an embodiment of a microclimate management system comprising a wearable garment or bodysuit configured to manage the temperature of the patient skin in contact with the wearable garment or bodysuit;

FIG. 2 is an illustration of an embodiment of a control unit configured to receive the environmental data from an environmental sensor unit and communicate with a blower, a centralized or localized data collection center or server, a computing device, and a smart phone;

FIG. 3 is an embodiment of an incontinence sensing system comprising a wearable garment or bodysuit configured to detect moisture in the wearable garment or bodysuit;

FIG. 4 is an illustration of an embodiment of a control unit configured to communicate with sensors, RFID tags, LEDs, a centralized or localized data collection center or server, a computing device, and a smart phone;

FIG. 5 is a first embodiment of a material comprising a disposable portion of the wearable garment or bodysuit of FIG. 3;

FIG. 6 is a first embodiment of a material comprising a disposable portion of the wearable garment or bodysuit of FIG. 3;

FIG. 7 is an embodiment of a movement monitoring system comprising a wearable garment or bodysuit that includes a movement detection system configured to detect movement in the wearable garment or bodysuit;

FIG. 8 is an embodiment of a wound monitoring or healing system comprising a wearable garment or bodysuit configured to detect and/or manage wound healing in a patient;

FIG. 9 is an illustration of an embodiment of a control unit configured to communicate with a sensor system of the wound monitoring or healing system of FIG. 7 a centralized or localized data collection center or server, a computing device, and a smart phone;

FIG. 10 is an embodiment of a tracking system comprising a wearable garment or bodysuit configured to track patient location and/or movement;

FIG. 11 is an illustration of an embodiment of a tracking device comprising a control unit and sensors configured to communicate with a centralized or localized data collection center or server, a computing device, and a smart phone;

FIG. 12 is one embodiment of a vitals monitoring system comprising a wearable garment or bodysuit and one or more sensors located in the wearable garment or bodysuit;

FIG. 13 is one embodiment of a light therapy system comprising a wearable garment or bodysuit and one or more lights and/or LEDS sewn in or embedded into the wearable garment or bodysuit;

FIG. 14 is one embodiment of a predictive AI system used in conjunction with the microclimate management system of FIG. 1, the incontinence sensing system of FIG. 3, the movement monitoring system of FIG. 7, the wound monitoring or healing system of FIG. 8, the tracking system of FIG. 10, the vitals monitoring system of FIG. 12, and the light therapy system of FIG. 13.

### DETAILED DESCRIPTION

A wearable garment or bodysuit according to the present disclosure, may be a gown or other sleepwear such as a hospital gown. The wearable garment or bodysuit may be a shirt with sleeves and/or a tie. Any data collected from any component of the wearable garment or bodysuit may be transmitted to a centralized or localized data collection center (e.g., ready connect). In some embodiments, the wearable garment or bodysuit may be a typical full gown that closes in the back. In other embodiments, the wearable garment or bodysuit may be a partial gown such as a skirt, shorts, pants, top only, vest, etc. In other embodiments, the wearable garment or bodysuit may be a blanket or a sheet. In other embodiments, the wearable garment or bodysuit may be an isolation tent, or a canopy positioned over the patient.

The wearable garment or bodysuit may include one or more technologies to improve patient care by measuring and/or assessing a patient condition, a patient therapy, or an environmental condition associated with the patient. For example, the wearable garment or bodysuit may include a microclimate management system, an incontinence sensing system, a movement monitoring system, a wound monitoring or healing system, a tracking system, a vitals monitoring system, a light therapy system and/or a predictive AI system.

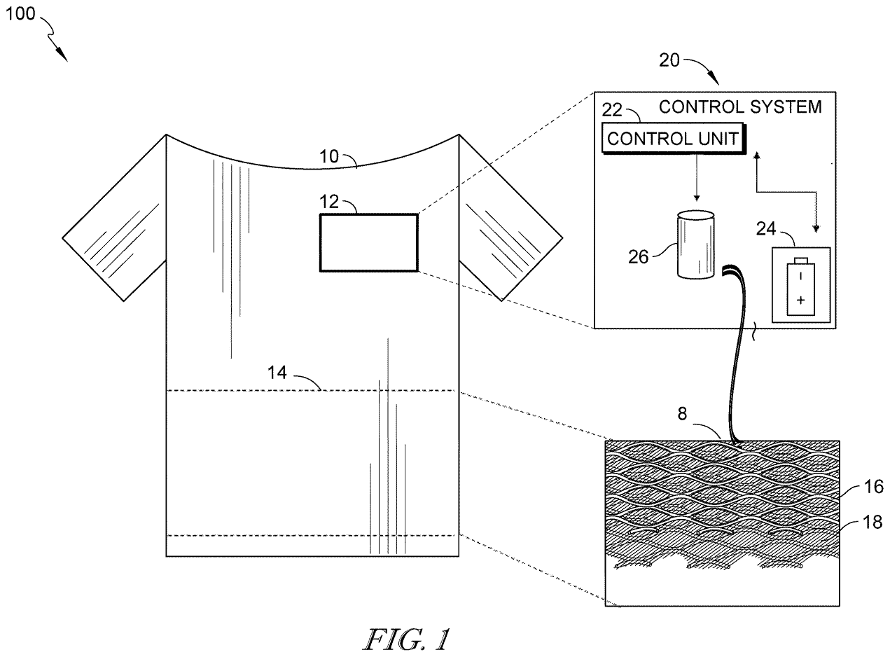

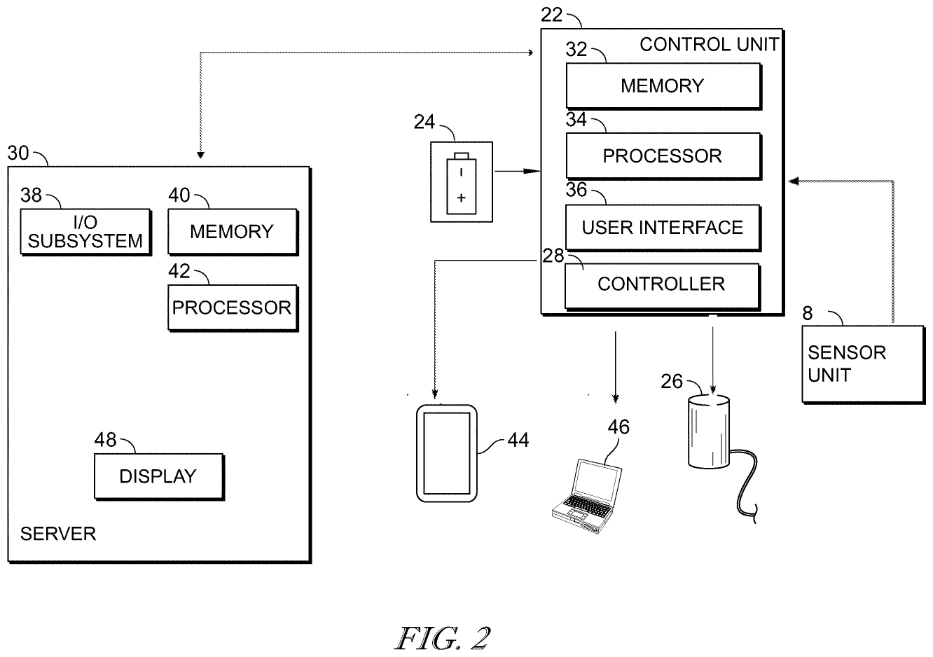

Referring to FIGS. 1 and 2, in the illustrative embodiment, a microclimate management system 100 comprises a wearable garment or bodysuit 10 configured to manage the temperature of the patient skin in contact with the wearable garment or bodysuit 10. The microclimate management system 100 includes a control system 20 (e.g., an air box 20) positioned in a reusable portion 12 of the wearable garment or bodysuit 10. The reusable portion 12 is a portion of the wearable garment or bodysuit 10 that is configured for multiple use by one or more patients. The control system 20 includes a control unit 22, a power source 24, and a blower 26. As shown in FIG. 2, the blower 26 is coupled to the control unit 22 for electrical communication with the control unit 22 and coupled to the wearable garment or bodysuit 10 for pneumatic communication with the wearable garment or bodysuit 10. In some embodiments, the blower 26 may be connected to a heater (not shown) configured to flow warm air to the wearable garment or bodysuit 10 and/or a cooler configured to flow cool air to the wearable garment or bodysuit 10. In some embodiments, the blower 26 may be connected to the control unit 22 by a wireless connection.

The blower 26 and/or the control unit 22 can be charged through the power source 24 (e.g., battery pack 24) of the control system 20. Alternatively, the blower 26 can be positioned on a patient support apparatus and charged in a docking station of the patient support apparatus. In some embodiments, the blower 26 may be wirelessly charged when located in the reusable portion 12. The operating parameters of the control system 20 may include blower speed settings and power settings. The wearable garment or bodysuit 10 may be consistent with the structures contemplated by the present disclosure is disclosed in U.S. Pat. No. 8,800,078, titled “LOCALIZED MICROCLIMATE MANAGEMENT” and in U.S. Pat. No. 9,370,847, titled “MICROCLIMATE SYSTEM FOR A PATIENT SUPPORT APPARATUS”, which are incorporated in their entirety.

The wearable garment or bodysuit 10 comprises a 3D spacer material 16 in a disposable portion 14 of the wearable garment or bodysuit 10. The disposable portion 14 is a portion of the wearable garment or bodysuit 10 that is configured for a single use or for use by only one patient. The disposable portion 14 is removably coupled to the reusable portion 12 of the wearable garment or bodysuit 10 such that the disposable portion 14 can be easily separated from the reusable portion 12. The 3D spacer material 16 is configured to allow air flow directly across the patient's skin through a plurality of air channels 18 for cooling and/or moisture management. The wearable garment or bodysuit 10 may include one or more zones comprising such 3D spacer material 16 and air channels 18 to target specific patient regions such as the sacral region. The wearable garment or bodysuit 10 may include heating for patient comfort. The microclimate management system 100 may include an environmental sensor unit 8 configured to detect environmental data corresponding to the environment surrounding the microclimate system.

The environmental sensor unit 8 is illustratively housed in the wearable garment or bodysuit 10. Alternatively, the environmental sensor unit 8 may be located along an intake path of the blower 26. In other embodiments, the environmental sensor unit 8 may be spaced apart from the intake path of the blower 26. The environmental sensor 8 may include a temperature sensor, a humidity sensor, and/or a pressure sensor. Each of the sensors included in the environmental sensor unit 8 may be configured to detect an environmental factor corresponding to the surroundings of the wearable garment or bodysuit 10. As shown in FIG. 2, each of the sensors 8 is coupled to the control unit 22 to communicate the detected environmental factors to the control unit 22. In some embodiments, the sensor 8 may be connected to the control unit 22 by a wireless connection.

In illustrative embodiments, as shown in FIG. 2, the control unit 22 is configured to receive the environmental data from the environmental sensor unit 8. A processor 34 in the control unit 22 is configured to determine if current operating parameters of the blower 26 provide a rated level of heat withdrawal and/or evaporative capacity through the wearable garment or bodysuit 10 based at least in part on the environmental data. The control unit 22 is configured to update the current operating parameters of the blower 26 if the current operating parameters do not provide the rated level of heat withdrawal and/or evaporative capacity through the wearable garment or bodysuit 10. A controller 28 is illustratively coupled to a user interface 36 to send and receive data to/from a user. In some embodiments, the processor 34 may activate a visual or an audible signal to a caregiver for activating the blower 26. In other embodiments, the processor 34 may communicate with the controller 28 to automatically activate the blower 26 based on the instructions stored in a memory module 32. Alternatively, the user may activate the blower 26 through the user interface 36 located in the control unit 22. Each of the components of the microclimate management system 100 may be connected to each other via a wired or a wireless connection. The control unit 22 is configured to communicate with a centralized or localized data collection center or server 30, a computing device 46, or a smart phone 44 by a wired or a wireless connection. The server 30 includes a processor 42, a memory module 40, an input/output system 38 and a display 48 configured to process and output processed data received from the control unit 22. Additionally, it is noted that the term “signal” as used in this disclosure means a waveform (e.g., electrical, optical, magnetic, mechanical or electromagnetic), such as DC, AC, sinusoidal-wave, triangular-wave, square-wave, vibration, and the like, capable of traveling through a medium.

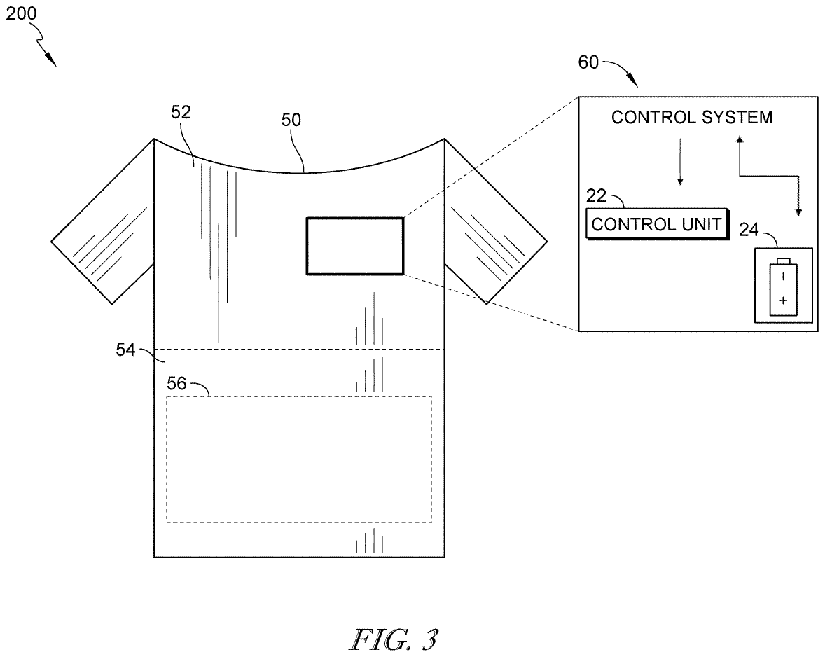

Referring to FIGS. 3-6, in the illustrative embodiment, an incontinence sensing system 200 comprises a wearable garment or bodysuit 50 configured to detect moisture. Referring to FIG. 3, the wearable garment or bodysuit 50 includes a reusable portion 52 that comprises a control system 60 and a disposable portion 54 that comprises material 56 and sensors 58. The disposable portion 54 is a portion of the wearable garment or bodysuit 50 that is configured for a single use or for use by only one patient. The reusable portion 52 is a portion of the wearable garment or bodysuit 50 that is configured for multiple use by one or more patients. The disposable portion 54 is removably coupled to the reusable portion 52 of the wearable garment or bodysuit 60 such that the disposable portion 54 can be easily separated from the reusable portion 52. The sensors 58 are configured to detect incontinence. The control system 60 includes the control unit 22 and the power source 24. The control unit 22 can be charged through the power source 24 (e.g., battery pack 24) located in the reusable portion 52. The wearable garment or bodysuit 50 may be consistent with the structures contemplated by the disclosure is disclosed in U.S. Pat. No. 11,020,284, titled “INCONTINENCE DETECTION PAD WITH LIQUID FILTER LAYER”, which is incorporated in its entirety.

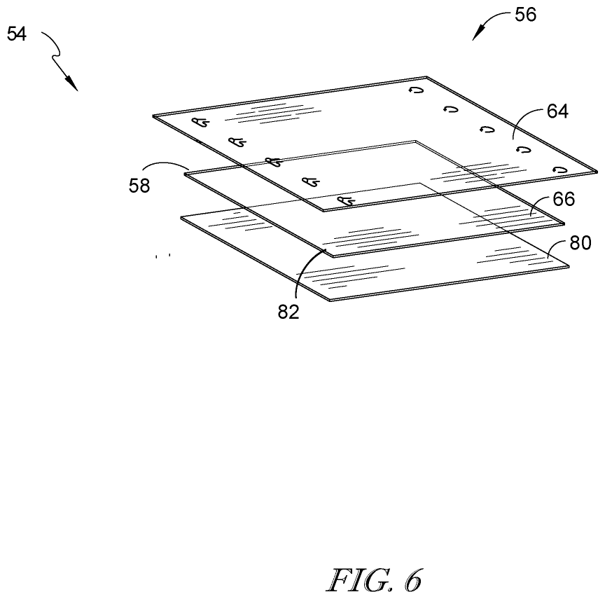

Referring to FIG. 6, in some embodiments, the material 56 in the wearable garment or bodysuit 50 includes a moisture absorbent core 66 that comprises a hydrogel layer 82 and the sensor 58. The sensor 58 is configured to detect moisture in the wearable garment or bodysuit 50 by measuring the absorbance of liquid in the hydrogel layer 82. The sensor 58 is configured to indicate the incontinence event to the caregiver. When the hydrogel layer 82 swells due to an increase in moisture, the sensor 58 is configured to detect the change in distance between a top layer 64 and a bottom layer 80 of the material 56 of the wearable garment or bodysuit 50. The sensor 58 is positioned in the disposable portion 54 of the wearable garment or bodysuit 50 and configured to indicate the incontinence event to the caregiver. The moisture absorbent core 66 may also keep the patient's skin dry after an incontinence event.

In some embodiments, the sensor 58 may comprise a color change indicator. The sensor 58 may change color after an incontinence event to signal the incontinence event to the caregiver. The color changing sensor 58 may be located in the disposable portion 54 of the wearable garment or bodysuit 50. The wearable garment or bodysuit 50 may include a new sensor 58 each time it is used.

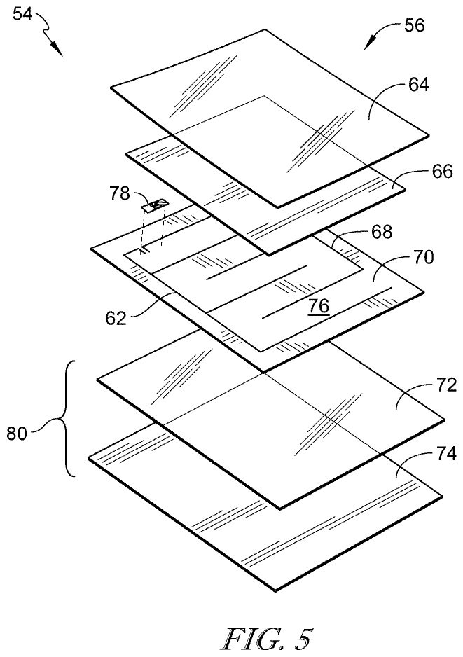

Referring to FIG. 5, in some embodiments, the material 56 of the disposable portion 54 of the wearable garment or bodysuit 50 includes, from top to bottom, the top layer 64, the moisture absorbent core 66 beneath the top layer 64, a passive RFID tag 78 beneath the moisture absorbent core 66, an insert layer 70 also positioned beneath the moisture absorbent core 66 and having electrodes 68, 62 printed on an electrical substrate 76, a first layer 72 of a bottom layer 80 beneath the insert layer 70, and a second layer 74 of the bottom layer 80 beneath the first layer 72 of the bottom layer 80. In some embodiments, the top layer 64 is made of a fluid permeable material and first layer 72 of bottom layer 80 is made of a fluid impermeable material. An adhesive may be used to couple the top layer 64, absorbent core 66, insert layer 70, and layers 72, 74 of the bottom layer 80 together. In some embodiments, the bottom layer 80 may comprise only the first layer 72. After fluid of a sufficient volume greater than a low volume threshold has passed through the absorbent core 66, an electrical pathway is formed between the electrodes 68, 62 by the fluid which enables the passive RFID tag 78 to emit a signal indicating an incontinence event has occurred in response to the passive RFID tag 78 being excited by external energy.

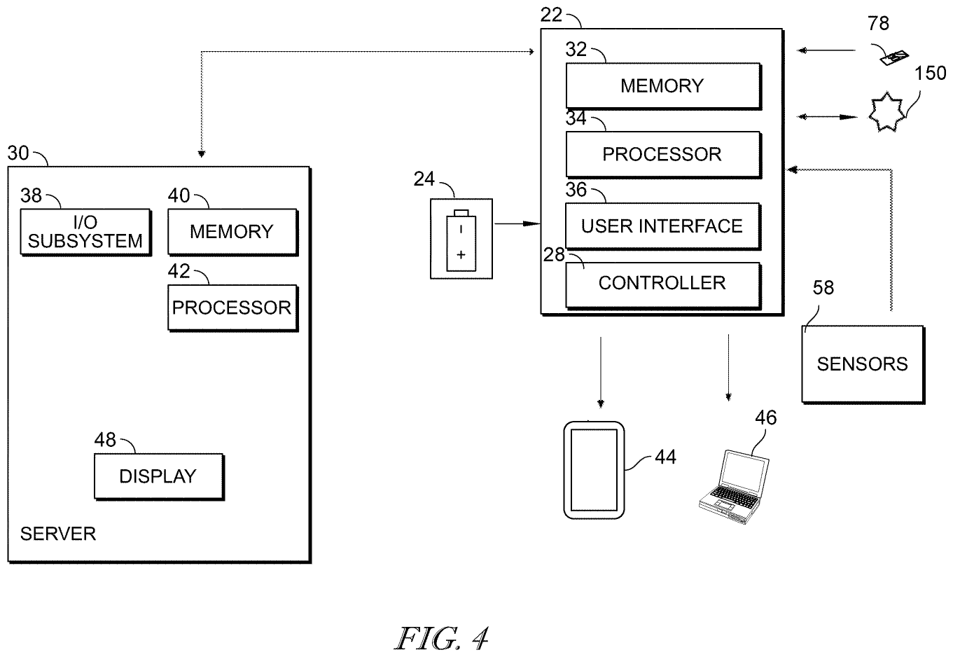

As illustrated in FIG. 4, the control unit 22 is configured to receive a signal from the sensor 58 or the RFID tag 78 and the processor 34 in the control unit 22 is configured to process the signal and indicate an incontinence event. As shown in FIG. 4 each of the sensors 58 or the RFID tag 78 is coupled to the control unit 22 to communicate the detected incontinence event to the control unit 22. In some embodiments, the sensor 58 or the RFID tag 78 may be connected to the control unit 22 by a wireless connection.

In some embodiments, the processor 34 in the control unit 22 is configured to process the signal and indicate an incontinence event if the change in distance between the top layer 64 and the bottom layer 80 is more than the baseline distance. In some embodiments, the processor 34 may be configured to activate a visual or an audible signal to a caregiver indicating an incontinence event. The control unit 22 is configured to communicate with a centralized or localized data collection center or server 30, a computing device 46, or a smart phone 44 with a wired or wireless connection. The server 30 includes a processor 42, a memory module 40, an input/output system 38 and a display 48 configured to process and output processed data received from the control unit 22.

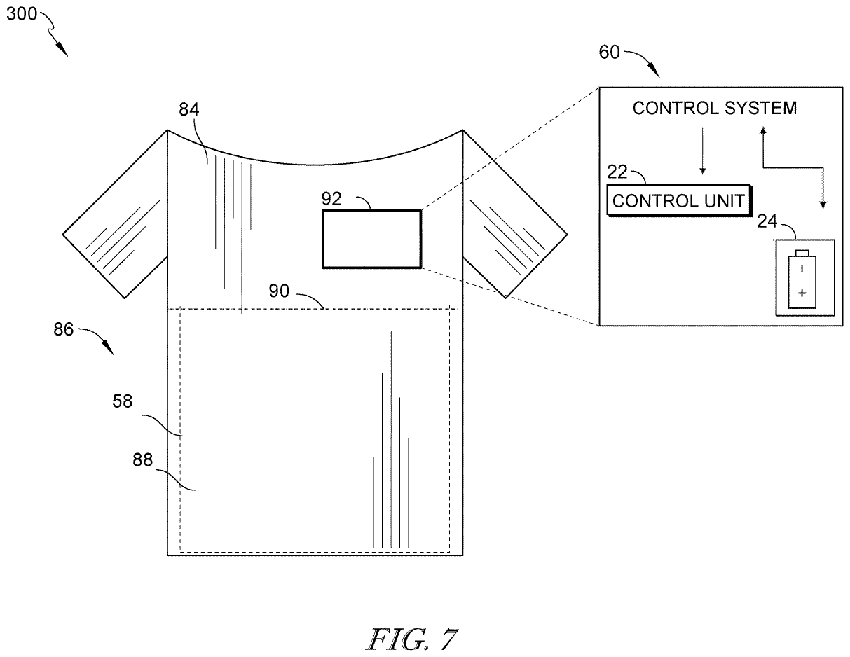

Referring to FIGS. 4 and 7, in the illustrative embodiment, a movement monitoring system 300 comprises a wearable garment or bodysuit 84 that includes a movement detection system 86 configured to detect movement in the wearable garment or bodysuit 84. The wearable garment or bodysuit 84 may be consistent with the structures contemplated by the disclosure is disclosed in U.S. Pat. No. 9,295,600, titled “PERSON SUPPORT APPARATUS WITH ACTIVITY AND MOBILITY SENSING”, which is incorporated in its entirety. In one embodiment, the movement detection system 86 may include a fabric 88 that responds to heat and/or voltage and/or sensors 58.

The movement detection system 86 may include one or more sensors 58 (e.g., voltage sensors and/or capacitive sensors) sewn into the wearable garment or bodysuit 84 in the elbow, knee, hip, and/or other areas where any voltage produced by movement of a particular part of patient body could be detected.

The wearable garment or bodysuit 84 includes a reusable portion 92 that comprises the control system 60 with the control unit 22 and the power source 24. The reusable portion 92 is a portion of the wearable garment or bodysuit 84 that is configured for multiple use by one or more patients. As shown in FIG. 4, the sensors 58 of the movement monitoring system 300 are configured to be connected via a wired or a wireless connection to the control unit 22 and the power source 24 located in the wearable garment or bodysuit 84. The control unit 22 is configured to receive a signal from the sensors 58 (e.g., voltage sensors and/or capacitive sensors). As shown in FIG. 4, the processor 34 in the control unit 22 is configured to process the signal and indicate any movement based on the voltage measured by the voltage sensors or capacitance measured by capacitive sensors and communicate with the server 30 via a wired or wireless connection. In some embodiments, the processor 34 may be configured to activate a visual or an audible signal to a caregiver indicating patient movement. The sensors 58 (e.g., voltage sensors and/or capacitive sensors) may be located in a disposable portion 90 of the wearable garment or bodysuit 84 or in the reusable portion 92 of the wearable garment or bodysuit 84. The disposable portion 90 is a portion of the wearable garment or bodysuit 84 that is configured for a single use or for use by only one patient. The disposable portion 90 is removably coupled to the reusable portion 92 of the wearable garment or bodysuit 84 such that the disposable portion 90 can be easily separated from the reusable portion 92. In some embodiments, the wearable garment or bodysuit 84 may entirely comprise of the reusable portion 92.

In other embodiments, the sensors 58 may comprise one or more accelerometers embedded into the fabric 88 of the wearable garment or bodysuit 84. The control unit 22 is configured to receive a signal from the accelerometers. The processor 34 in the control unit 22 is configured to process the signal and indicate any movement based on the acceleration measured by the accelerometers. In some embodiments, the processor 34 may be configured to activate a visual or an audible signal to a caregiver indicating patient movement.

In other embodiments, the sensors 58 may be pressure sensors positioned in one or more air cells. The sensors 58 may be configured to detect differences in pressure in each air cell to determine any change in patient movement. The pressure sensors may also provide pressure readings at the patient interface with the wearable garment or bodysuit 84. The control unit 22 may be configured to receive a signal from the pressure sensors. The processor 34 in the control unit 22 may be configured to process the signal and indicate any movement based on the pressure changes determined by the pressure sensors. In some embodiments, the processor 34 may be configured to activate a visual or an audible signal to a caregiver indicating patient movement.

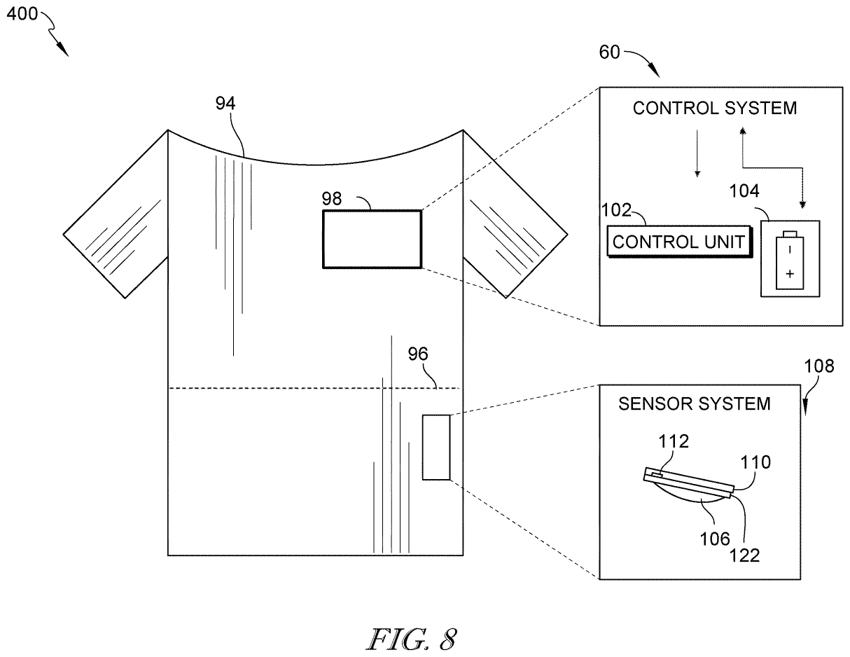

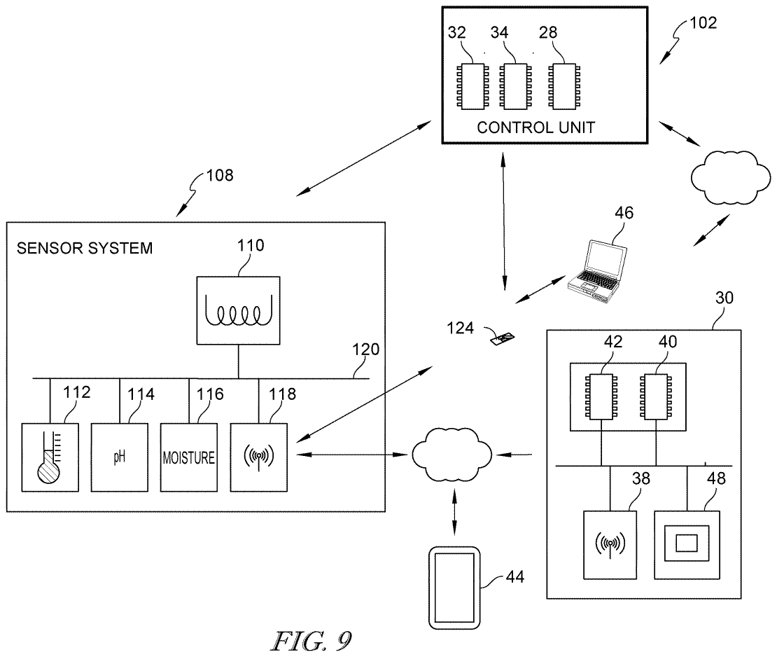

Referring to FIGS. 8 and 9, in the illustrative embodiment, a wound monitoring or healing system 400 comprises a wearable garment or bodysuit 94 configured to detect and/or manage wound healing in a patient. The wearable garment or bodysuit 94 comprising a sensor system 108 on a disposable portion 96 of the wearable garment or bodysuit 94 may prevent the occurrence of a wound and/or may monitor the healing of an existing wound. The disposable portion 96 is a portion of the wearable garment or bodysuit 94 that is configured for a single use or for use by only one patient. The wearable garment or bodysuit 94 may be consistent with the structures contemplated by the disclosure is disclosed in US Publication No. 2019/0060126, titled “SYSTEMS FOR MONITORING WOUNDS AND WOUND DRESSING STATUS AND SYSTEMS FOR PROTECTING WOUNDS”, which is incorporated in its entirety.

The sensor system 108 that can detect temperature, blood flow, oxygen level, moisture content, pH, and/or depth of wound embedded in the wearable garment or bodysuit 94. The sensor system 108 includes a wound dressing 122, a heating element 110, and a temperature sensor 112 covering a wound 106. The wound dressing 122 is configured to cover the wound surface of the wound 106. The wound dressing 122 may seal the wound surface of the wound 106 in order to protect the wound surface from external pathogens. The wound dressing 122 includes a wound facing surface that faces the wound surface of the wound 106 and an outer surface. The wound dressing 122 may include, for example, moisture-retentive foam, film, hydrogel, hydrocolloid, or alginate dressings, biologics, skin substitutes, and specifically including dressings that comprise a negative pressure wound therapy (NPWT) system.

The heating element 110 is coupled to the outer surface of the wound dressing 122. In some embodiments, the heating element 110 may be coupled to the wound facing surface of the wound dressing 122. The heating element 110 is configured to heat the wound 106 of a patient and maintain the wound 106 at a predetermined temperature (e.g., 37 degrees Celsius) or a predetermined temperature range (e.g., 36 degrees Celsius through 38 degrees Celsius). The temperature sensor 112 is configured to detect the temperature of the wound 106. The temperature sensor 112 is placed on the outer surface of the wound dressing 122, the temperature sensor 112 may be placed at the wound facing surface of the wound dressing 122. The temperature sensor 112 may communicate the measured temperature to a control unit 102 of the control system 60 located in a reusable portion 98 of the wearable garment or bodysuit 94. The reusable portion 98 is a portion of the wearable garment or bodysuit 94 that is configured for multiple by one or more patients. The disposable portion 96 is removably coupled to the reusable portion 98 of the wearable garment or bodysuit 94 such that the disposable portion 96 can be easily separated from the reusable portion 98. The heating element 110 is powered by a power source 104 located in the reusable portion 98. In some embodiments, the control unit 102 and/or the power source 104 may be located in the sensor system 108.

In some embodiments, as shown in FIG. 9, the sensor system 108 may include the heating element 110, the temperature sensor 112, a pH sensor 114, and/or a moisture sensor 116. Some embodiments of the sensor system 108 may include a network interface hardware 118. In some embodiments, the sensor system 108 may comprise an infrared or doppler laser (not shown).

As shown in FIG. 9, a communication path 120 may communicatively couple different components of the sensor system 108. Each of components may operate as a node that may send and/or receive data from the control unit 102 either via wired or wireless communication. As used herein, the term “communicatively coupled” means that coupled components are capable of exchanging data signals with one another such as, for example, electrical signals via conductive medium, electromagnetic signals via air, optical signals via optical waveguides, and the like.

The control unit 22 is configured to receive a temperature detected by the temperature sensor 112 and control the operation of the heating element 110 based on the detected temperature. Signals from one or more sensors 112, 114, 116 located on the sensor system 108 are transmitted to the control unit 102. The processor 34 in the control unit 102 is configured to process the signal and indicate status of wound healing based on the measurements made by the sensors 112, 114, 116 and data stored in the memory modules 32. The controller 28 is configured to control the heating element 110 based on the measurements made by the sensors 112, 114, 116 and data stored in the memory modules 32. In some embodiments, the processor 34 may activate a visual or an audible signal to a caregiver indicating wound status. The network interface hardware 118 and/or the control unit 102 can be communicatively coupled to the communication path 120 and can be any device capable of transmitting and/or receiving data to and from an external device such as a RFID reader 124 coupled to a computing device 46, a remote server 30, or a smart phone 44.

In some embodiments, the wearable garment or bodysuit 94 may be modified to include a patch of foam or a softer material to lessen the impact of the wearable garment or bodysuit 94 on the skin in areas where wounds 106 are more likely to occur. Such modifications can keep the area prone to wounds dry, spread a load over a larger area, and prevent abrasion from patient or surface movement.

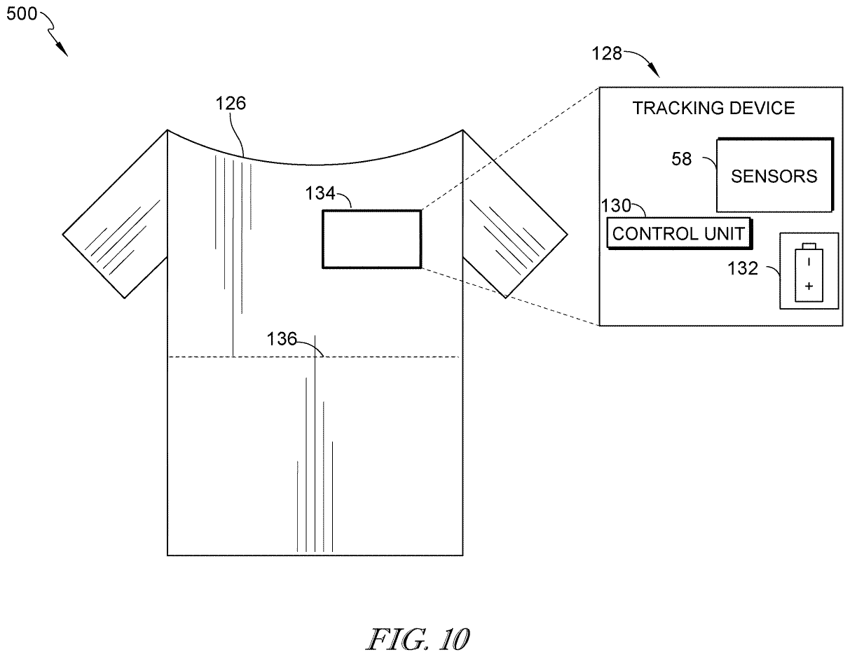

Referring to FIG. 10, in the illustrative embodiment, a tracking system 500 comprises a control system 128 (e.g., a tracking device 128) that is configured to connect to a centralized data system, a nurse call system and/or other systems in a wearable garment or bodysuit 126. Patient tracking is helpful when the patient leaves the patient room for therapy or visits a medical or scanning lab or if the patient is a flight risk. The tracking device 128 includes one or more sensors 58 and may be located on one or more components of the wearable garment or bodysuit 126. For example, the tracking device 128 may be located on a wrist band, an adhesive patch, an ear bud, an ankle bracelet, or a head covering. The tracking device 128 may be sewn or welded into the wearable garment or bodysuit 126. The tracking device 128 may be located in a reusable portion 134 of the wearable garment or bodysuit 126. The reusable portion 134 is a portion of the wearable garment or bodysuit 126 that is configured for multiple by one or more patients.

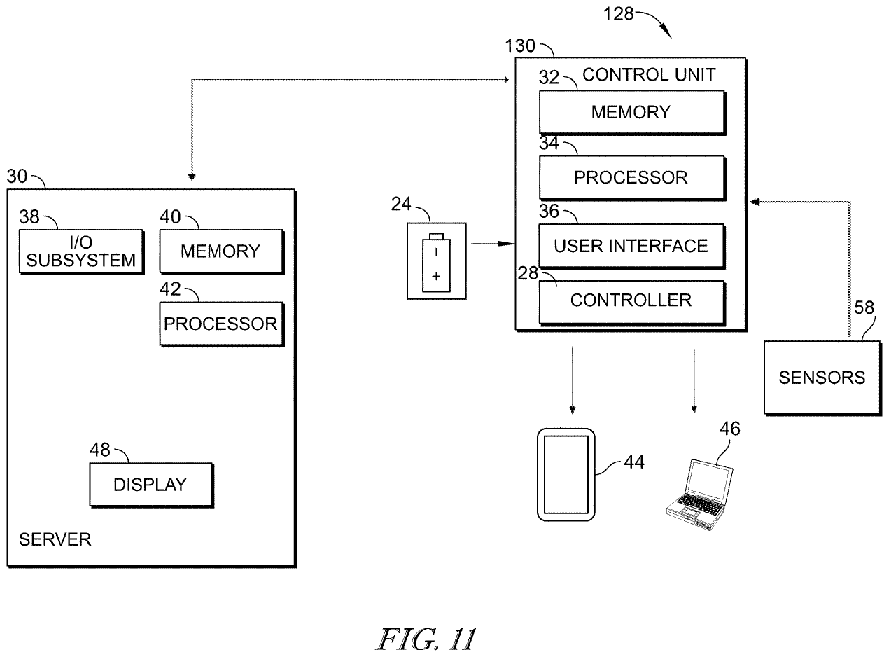

As shown in FIGS. 10 and 11, the tracking device 128 comprises a control unit 130 and a power source 132 and the control unit 130 is configured to be connected via a wired or a wireless connection to the computing device 46, the remote server 30, or the smart phone 44. The server 30 is configured to receive a signal from the control unit 130 of the tracking device 128. The processor 42 in the server 30 is configured to process the signal based on instructions stored in the memory module 40 and display the location of the patient on the display 48. The computing device 46 and/or the smart phone 44 may similarly process and/or display data obtained from the control unit 130. In some embodiments, the tracking system 500 may include sensors 58 located in different components of the wearable garment or bodysuit 126. For example, the sensors 58 may be located in a disposable portion 136 of the wearable garment or bodysuit 126. The disposable portion 136 is a portion of the wearable garment or bodysuit 126 that is configured for a single use or for use by only one patient. The disposable portion 136 is removably coupled to the reusable portion 134 of the wearable garment or bodysuit 126 such that the disposable portion 136 can be easily separated from the reusable portion 134. As shown in FIG. 11, each of the sensors 58 is coupled to the control unit 22 to communicate patient location to the control unit 22. In some embodiments, the sensor 58 may be connected to the control unit 22 by a wireless connection. The control unit 130 is configured to receive location signals from the sensors 58 and communicate with the computing device 46, the remote server 30, or the smart phone 44 by a wired or a wireless connection.

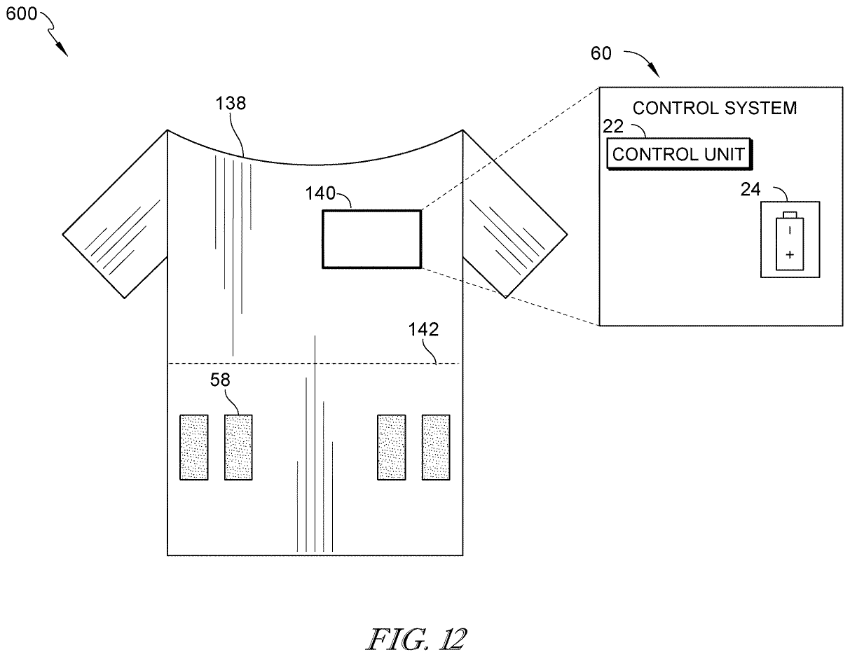

Referring to FIGS. 4 and 12, in the illustrative embodiment, a vitals monitoring system 600 comprises one or more sensors 58 located on one or more components of a wearable garment or bodysuit 138. For example, the sensors 58 may be located on a wrist band, an adhesive patch, an ear bud, an ankle bracelet, or a head covering. The sensors 58 may include one or more of a temperature sensor, a moisture sensor, a pH sensor, heart rate monitor, or an oxygen monitor.

The sensors 58 are configured to be connected via a wired or a wireless connection to the power source 24 of the control system 60 located in a reusable portion 140 of the wearable garment or bodysuit 138. The reusable portion 140 is a portion of the wearable garment or bodysuit 138 that is configured for multiple use by one or more patients. The sensors 58 are configured to be connected via a wired or a wireless connection to the control unit 22 located in the reusable portion 140 of the wearable garment or bodysuit 138. As shown in FIG. 4, the control unit 22 is configured to receive a signal from the sensors 58. The processor 34 in the control unit 22 is configured to process the signal and indicate patient status based on the measurements made by the sensors 58 and instructions stored in the memory module 32. The control unit 22 is configured to be connected via a wired or a wireless connection to the computing device 46, the remote server 30, or the smart phone 44. In some embodiments, the processor 34 may be configured to activate a visual or an audible signal to a caregiver indicating patient status. The sensors 58 are located in a disposable portion 142 of the of the wearable garment or bodysuit 138. The disposable portion 142 is a portion of the wearable garment or bodysuit 138 that is configured for a single use or for use by only one patient. The disposable portion 142 is removably coupled to the reusable portion 140 of the wearable garment or bodysuit 138 such that the disposable portion 142 can be easily separated from the reusable portion 140.

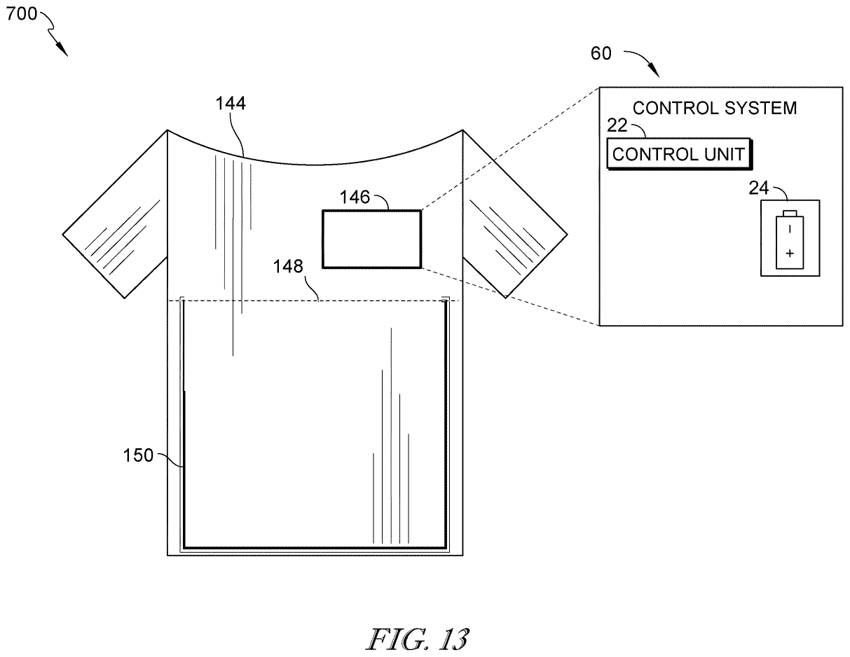

Referring to FIGS. 4 and 13, in the illustrative embodiment, a light therapy system 700 can be used to improve the healing of cells in the patient body. Certain wavelengths of light can have different beneficial effects on the body. The light therapy system 700 comprises one or more lights and/or LEDs 150 that are sewn in or embedded into a wearable garment or bodysuit 144. The LEDs 150 are sewn in or embedded into a disposable portion 148 of the wearable garment or bodysuit 144. The disposable portion 148 is a portion of the wearable garment or bodysuit 144 that is configured for a single use or for use by only one patient. In some embodiments, LEDs 150 are sewn in or embedded into a reusable portion 146 of the wearable garment or bodysuit 144. The reusable portion 146 is a portion of the wearable garment or bodysuit 144 that is configured for multiple use by one or more patients. The disposable portion 148 is removably coupled to the reusable portion 146 of the wearable garment or bodysuit 144 such that the disposable portion 148 can be easily separated from the reusable portion 146. The one or more lights and/or LEDs 150 may provide light of the same wavelength or may provide light of different wavelengths.

The lights and/or LEDs 150 are configured to be connected via a wired or a wireless connection to the power source 24 located in the reusable portion 146 of the wearable garment or bodysuit 144. The LEDs 150 are configured to be connected via a wired or a wireless connection to the control unit 22 of the control system 60 located in the reusable portion 146 of the wearable garment or bodysuit 144. As shown in FIG. 4, the control unit 22 is configured to receive a signal from the lights and/or LEDs 150. The processor 34 in the control unit 22 is configured to process the signal and indicate a light therapy status of the patient based on instructions stored in the memory module 32. The control unit 22 is configured to be connected via a wired or a wireless connection to the computing device 46, the remote server 30, or the smart phone 44. In some embodiments, the patient may activate the lights and/or LEDs 150 to follow treatment protocols by using the user interface 36. In some embodiments, the processor 34 may be configured to activate a visual or an audible signal to a caregiver indicating patient status.

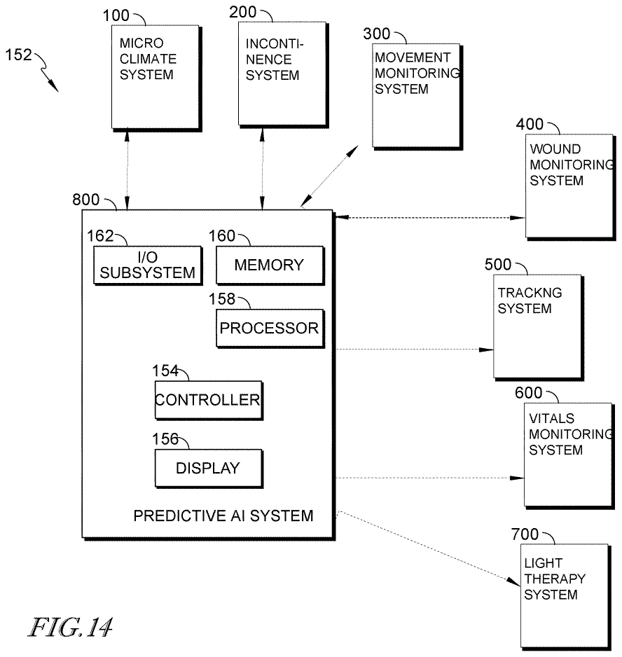

In one embodiment, as illustrated in FIG. 14, a predictive AI system 800 can be used in conjunction with one or more of the microclimate management system 100, the incontinence sensing system 200, the movement monitoring system 300, the wound monitoring or healing system 400, the tracking system 500, the vitals monitoring system 600, and/or the light therapy system 700. A wearable garment or bodysuit 152 comprising the predictive AI system 800 is configured to collect data indicative of the symptoms of the patient's medical conditions. The wearable garment or bodysuit 152 comprising the predictive AI system 800 is configured to predict the patient's symptoms before they occur. For example, in some embodiments, the wearable garment or bodysuit 152 comprising the predictive AI system 800 is configured to predict pressure injuries, patient falls, incontinence, sepsis, stroke, cardiac arrest, etc. before they occur. The predictive AI system 800 is configured to receive a signal from one or more of the microclimate management system 100, the incontinence sensing system 200, the movement monitoring system 300, the wound monitoring or healing system 400, the tracking system 500, the vitals monitoring system 600, and/or the light therapy system 700. A processor 158 is configured to process the signal and predict the patient's symptoms before they occur based on instructions stored in a memory module 160. A controller 154 is configured to be operable in response to the processor 158. In some embodiments, the processor 158 may activate a visual signal on a display 156 or an audible signal to a caregiver indicating patient status prediction. A caregiver may be able to access data through an input/output system 162. In some embodiments, the predictive AI system 800 may be connected via a wired or a wireless connection to the computing device 46, the remote server 30, or the smart phone 44.

Although this disclosure refers to specific embodiments, the disclosure is not limited to the disclosed embodiments. It will be understood by those skilled in the art that various changes in form and detail can be made without departing from the subject matter set forth in the accompanying claims. For example, the disclosure has been illustrated and described in detail in the drawings and the foregoing description, such illustration and description are to be considered illustrative or exemplary and not restrictive. From reading the present disclosure, other modifications will be apparent to a person skilled in the art. Such modifications may involve other features, which are already known in the art and may be used instead of or in addition to features already described herein. In the claims, the word “comprising” does not exclude other elements or steps, and the indefinite article “a” or “an” does not exclude a plurality.

1. A garment comprising:
a disposable portion, the disposable portion comprising a monitoring system having a sensor system configured to provide a signal,
a reusable portion, the reusable portion comprising a control system having a control unit with a processor configured to receive and analyze the signal from the disposable portion and a power source configured to power the control unit, and
wherein the monitoring system is configured to provide data about a patient condition, a patient therapy, or an environmental condition associated with a patient.

2. The garment of claim 1, wherein the sensor system is charged by the power source located in the reusable portion of the garment.

3. The garment of claim 1, wherein the sensor system is configured to provide microclimate environment data to the control unit, and wherein the control unit is configured to direct a blower connected to the control unit to blow air through a plurality of air channels in the disposable portion of the garment.

4. The garment of claim 3, wherein the disposable portion comprises a 3D spacer material configured to create the plurality of air channels.

5. The garment of claim 3, wherein the blower is configured to blow hot air or cold air through the plurality of air channels.

6. The garment of claim 3, further comprising a plurality of zones, wherein the blower is configured to blow air through one or more zones.

7. The garment of claim 1, wherein the sensor system is a wound sensor system configured to provide wound healing data to the control unit, and wherein the control unit is configured to direct a heating element located in the wound sensor system.

8. The garment of claim 7, wherein the wound sensor system is configured to measure temperature, blood flow, oxygen level, moisture content, pH, or depth of a wound.

9. The garment of claim 7, wherein the wound sensor system comprises the heating element positioned on a wound facing side of a wound dressing.

10. The garment of claim 7, wherein the disposable portion comprises one or more patches of foam configured to minimize abrasion in zones that cover parts of a patient body prone to wounds.

11. A garment comprising:
a disposable portion, the disposable portion comprising a sensor system configured to provide a signal,
a reusable portion, the reusable portion comprising a control system having a control unit with a processor configured to receive and analyze the signal from the disposable portion and a power source configured to power the control unit, and
wherein the sensor system is configured to provide data to a predictive AI system.

12. The garment of claim 11, wherein the data is about a patient condition, a patient therapy, or an environmental condition associated with a patient.

13. The garment of claim 12, wherein the sensor system is configured to provide microclimate environment data to the control unit, and wherein the control unit is configured to direct a blower connected to the control unit to blow air through a plurality of air channels in the disposable portion of the garment.

14. The garment of claim 12, wherein the sensor system is a wound sensor system configured to provide wound healing data to the control unit, and wherein the control unit is configured to direct a heating element located in the wound sensor system.

15. The garment of claim 12, wherein the disposable portion comprises a first layer separated from a second layer by a layer of hydrogel.

16. The garment of claim 15, wherein the sensor system is configured to measure a height of the layer of hydrogel.

17. A method of acquiring data about a patient condition, a patient therapy, or an environmental condition associated with a patient comprising:
monitoring the patient wearing a garment, the garment including a disposable portion comprising a monitoring system having a sensor system, and a reusable portion comprising a control system including a control unit and a power source,
acquiring a signal from the sensor system located on the garment,
processing the signal in the control system with a processor configured to receive and analyze the signal, and
outputting processed data to a caregiver.

18. The method of claim 17, wherein the sensor system is configured to provide microclimate environment data to the control unit, and wherein the control unit is configured to direct a blower connected to the control unit to blow air through a plurality of air channels in the disposable portion of the garment.

19. The method of claim 17, comprising a movement monitoring system that includes the sensor system in a movement detection system configured to provide movement associated data with at least one part of patient body.

20. The method of claim 19, wherein the movement detection system comprises a fabric that responds to heat or voltage.
